(19) United States
(12) Patent Application Publication
(10) Pub. No.: US 2024/0400472 A1
(43) Pub. Date: Dec. 5, 2024

(54) PROCESS FOR PRODUCING RENEWABLE ALKYLBENZENE PRODUCTS

(71) Applicant: UOP LLC, Des Plaines, IL (US)

(72) Inventors: Phuong T.M. Do, Mount Prospect, IL (US); Hai Du, Hinsdale, IL (US); Geoffrey W. Fichtl, Chicago, IL (US); James T. Wexler, Wheaton, IL (US); Eseoghene Jeroro, Chicago, IL (US); Patrick C. Whitchurch, Elgin, IL (US)

(21) Appl. No.: 18/500,161

(22) Filed: Nov. 2, 2023

Related U.S. Application Data
(60) Provisional application No. 63/504,879, filed on May 30, 2023.

(51) Int. Cl. C07C 1/207

(52) U.S. Cl. CPC C07C 1/2078

(57) ABSTRACT

Process for producing alkylbenzenes from triglycerides, in particular triglycerides having 60% or more of alkyl chains having less than 16 carbon atoms are described. The process include a linear selective cracking process to crack C14+ chains into C9 to C14 chains which are useful for making linear alkylbenzene for use in detergents.

### RELATED APPLICATIONS

This application claims priority to U.S. Provisional Patent Application Ser. No. 63/504,879 filed on May 30, 2023, the entirety of which is incorporated herein by reference.

### BACKGROUND

Linear alkylbenzenes are organic compounds with the formula C₆H₅CₙH₂ₙ₊₁. While the alkyl carbon number, “n” can have any practical value, detergent manufacturers desire that alkylbenzenes have alkyl carbon number in the range of 9 to 16 and preferably in the range of 9 to 14 These specific ranges are often required when the alkylbenzenes are used as intermediates in the production of surfactants for detergents. The alkyl carbon number in the range of 9 to 14 falls in line with the specifications of the detergents industry.

Because the surfactants created from alkylbenzenes are biodegradable, the production of alkylbenzenes has grown rapidly since their initial uses in detergent production in the 1960s. The linearity of the paraffin chain in the alkylbenzenes is key to the material's biodegradability and effectiveness as a detergent. A major factor in the final linearity of the alkylbenzenes is the linearity of the paraffin component.

While detergents made utilizing alkylbenzene-based surfactants are biodegradable, previous processes for creating alkylbenzenes are not based on renewable sources. Specifically, alkylbenzenes are currently produced from kerosene refined from crude extracted from the earth. Due to the growing environmental prejudice against fossil fuel extraction and economic concerns over exhausting fossil fuel deposits, there may be support for using an alternate source for biodegradable surfactants in detergents and in other industries.

Accordingly, it is desirable to provide linear alkylbenzenes with a high degree of linearity that are made from biorenewable sources instead of being extracted from the earth. Further, it is desirable to provide renewable linear alkylbenzenes from easily processed triglycerides and fatty acids from vegetable, animal, nut, and/or seed oils. Palm kernel oil, coconut oil and babassu oil have a composition that is high in the desirable range of C9-C14 n-paraffins that aligns with the alkyl carbon number range desired of the detergent industry. Such renewable sources also have a high amount of nC16 to nC18 feeds, and it is desirable to convert those feeds to nC9 to nC14 feeds with a high per-pass yield. These nC9 to nCl4 intermediate products are useful in eventually making linear alkylbenzene types of detergents through additional process steps. It is further desirable that the resulting nC9 to nC14 paraffins are linear products with a minimum of branched isomer products.

### BRIEF DESCRIPTION OF THE DRAWINGS

FIG. 1 is a schematic view of one embodiment of a process for producing alkylbenzenes from triglycerides according to the present invention.

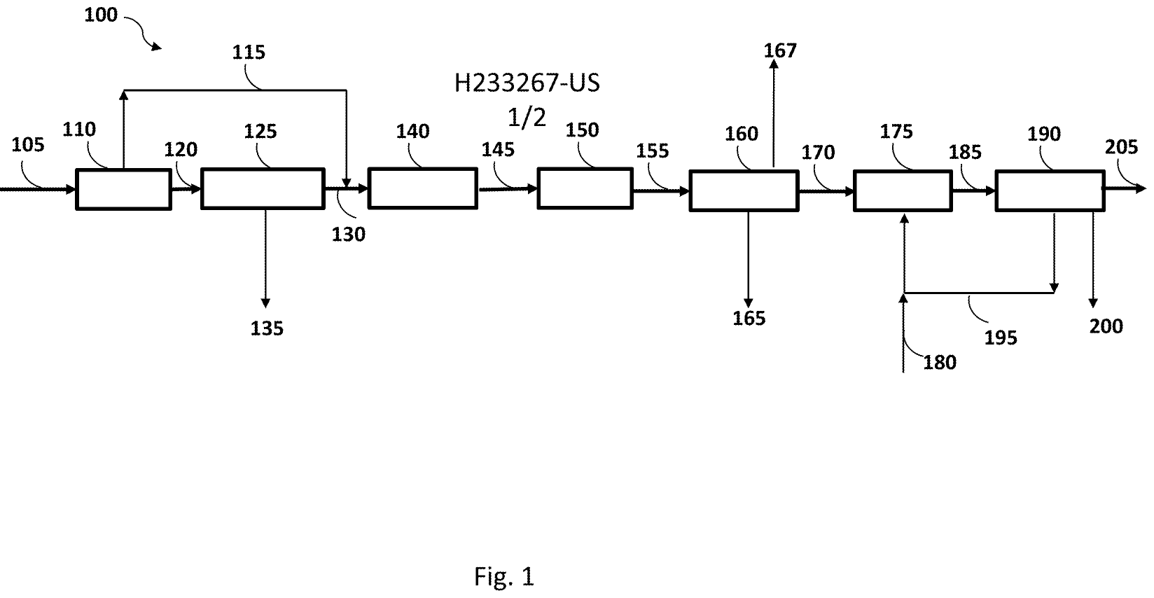

FIG. 2 is a plot of the mass-% normal paraffins versus deoxygenation temperature in accordance with Example 2.

### DETAILED DESCRIPTION

The present invention relates to a process for producing alkylbenzenes from triglycerides, in particular triglycerides producing 60 wt. % or more of normal paraffins having less than 16 carbon atoms after hydrogenation. Some of these triglycerides also produce a substantial amount of normal paraffins having 16 to 24 carbon atoms, e.g., 20 to 30%, after dehydrogenation. These paraffins are longer than desired for the production of detergent products.

In some embodiments, the amount of normal paraffins having 16 carbon atoms after dihydrogenation is less than 20%, or less than 15%, or less than 10%.

The triglycerides come from natural oils. Natural oils are not based on kerosene or other fossil fuels. Natural oils include those derived from plant or algal material or animal fats, nut, and/or seed oils, and triglyceride-containing oils, and are often referred to as renewable oils. Natural oils typically comprise triglycerides, free fatty acids, or combinations thereof. Natural oils include, but are not limited to, Arachis oil (peanut oil; groundnut oil), Babassu oil, Coconut oil, Cottonseed oil, Grapeseed oil, Maize oil (corn oil), Mustard seed oil, Palm kernel oil, Palm oil, Palm olein (the liquid fraction derived from the fractionation of palm oil), Palm stearin (the high-melting fraction derived from the fractionation of palm oil), Rapeseed oil, Rapeseed oil—low erucic acid (low erucic acid turnip rape oil; low erucic acid colza oil; canola oil), Safflowerseed oil (safflower oil; carthamus oil; kurdee oil), Safflowerseed oil—high oleic acid (high oleic acid safflower oil; high oleic acid carthamus oil; high oleic acid kurdee oil), Sesameseed oil (sesame oil; gingelly oil; benne oil; ben oil; till oil; tillie oil), Soya bean oil (soybean oil), Sunflowerseed oil (sunflower oil), and Sunflowerseed oil—high oleic acid (high oleic acid sunflower oil).

In some embodiments, the triglyceride produces no less than 15 wt. % of normal paraffins having 12 or 14 carbon atoms after deoxygenation. In some embodiments, the triglyceride produces no less than 10 wt. % of normal paraffins having 12 carbon atoms after deoxygenation.

The process for making alkylbenzenes from triglycerides according to the present invention involves the deoxygenation of the triglycerides to form paraffins. The paraffins are separated (by fractionation, distillation, and the like) into a C9 to C14 stream comprising C9 to C14 paraffins and a C14+ stream comprising C14+(i.e., containing carbon chains from C15 to C28) paraffins. The C14+ stream is sent to a separate linear selective cracking unit to crack the C14+ paraffins; the cracked paraffins are fractionated into a first stream comprising the C9 to C14 normal and lightly branched paraffins and a second stream comprising isoparaffins. Contaminants, including but not limited to, comprise sulfur compounds, or nitrogen compounds, or phosphorous compounds, or oxygenates, or aromatics or combinations thereof, are removed from the C9 to C14 stream and the first stream. The decontaminated stream is dehydrogenated to form olefins, di-olefins, and aromatics. The di-olefins are selectively hydrogenated to form additional olefins, and the aromatics are separated and removed forming an aromatics stream comprising the aromatics and a mono-olefin stream comprising the mono-olefins. Benzene is alkylated with the olefins, and the alkylation effluent comprises alkylbenzenes and benzene. The alkylbenzenes are then isolated.

The linear selective cracking step will be further described. The linear selective cracking takes place in a separate unit, rather than in the bottom bed of a first stage hydrocracking reactor because sulfur and nitrogen contaminants from the first stage can poison a metal-based hydrocracking catalyst. The C14+ paraffins are selectively cracked over the C9 to C14 due to higher absorption energy.

Selection of particular metal catalysts, including noble metals (such as ruthenium and platinum), and nickel can produce a much higher yield of normal paraffins with 9-14 carbons than previous processes. Suitable catalysts include, but are not limited to, Ru/ZrO₂, a Pt—Al₂O₃, Ni-alumina, or a NiOₓ/clay. With these catalysts, the C14+ stream can generate linear cracking products without significant amounts of branched isomer production.

Of the preferred catalysts, the Ru catalyst exhibits much higher activity and per-pass nC9 to nC14 yield than the other catalysts. Under the optimized reaction conditions, it also produces very small amounts of methane and isomerized product. This has been found to be the best catalyst for such chemical transformation process. The Pt-Al₂O₃ catalyst can produce even lower methane yield than the Ru based catalyst with slightly less linear product yield.

To limit catalyst deactivation, the feed is treated to remove sulfur, chloride and metal contamination before hydrodeoxygenation. Otherwise, sulfur, chloride and metals accumulate on the catalyst, and this leads to deactivation. A high temperature hydrogen treatment was shown to recover some of the lost activity. The degree of hydrodeoxygenation can affect the selectivity to each of the normal paraffins in the 9 to 14 carbon range. A large degree of hydrodeoxygenation can bias the hydrodeoxygenated composition largely in favor of normal dodecane and normal decane to the detriment of normal undecane and normal tridecane. A small degree of hydrodeoxygenation can bias the hydrodeoxygenated composition in favor of normal undecane and normal tridecane to the detriment of normal dodecane and normal decane.

The hydrodeoxygenation reactor temperatures are kept low, less than 343° C. (650° F.) for typical biorenewable feedstocks and less than 304° C. (580° F.) for feedstocks with higher free fatty acid (FFA) concentration to avoid polymerization of olefins found in FFA. Generally, hydrodeoxygenation reactor pressure of about 700 kPa (100 psig) to about 21 MPa (3000 psig) are suitable.

The overall process will now be described.

The linearity of the alkylbenzene product is mostly dependent on the linearity of the paraffins used to alkylate the benzene. It is a common rule of thumb by those skilled in the art that the linearity of a paraffin feed drops by about 5-7 mass % after dehydrogenation and alkylation. Therefore, paraffin with 97 mass % linearity (or alternatively 3 mass % isoparaffin) would result in an alkylbenzene product with linearity around 90-92 mass %. This sets the requirement for paraffin linearity about 5-7 mass % higher than the specification for the alkylbenzene product. Typically the linearity of the paraffin product is measured by UOP 621, UOP411, or UOP732 standard test method available from ASTM, which is hereby incorporated by reference in its entirety. Linear alkylbenzenes may be analyzed using ASTM Standard Test Method D4337 hereby incorporated by reference in its entirety.

In the Figure, an exemplary system 100 for producing an alkylbenzene product from a specific triglyceride feed is illustrated.

In the illustrated embodiment, the selected triglyceride feed 105 is delivered to a deoxygenation unit 110 which also receives a hydrogen feed (not shown). In the deoxygenation unit 110, the fatty acids in the selected triglyceride feed 105 are deoxygenated and converted into normal paraffins. Structurally, triglycerides are formed by three, typically different, fatty acid molecules that are bonded together with a glycerol bridge. The glycerol molecule includes three hydroxyl groups (HO—) and each fatty acid molecule has a carboxyl group (COOH). In triglycerides, the hydroxyl groups of the glycerol join the carboxyl groups of the fatty acids to form ester bonds. Therefore, during deoxygenation, the fatty acids are freed from the triglyceride structure and are converted into normal paraffins. The glycerol is converted into propane, and the oxygen in the hydroxyl and carboxyl groups is converted into water, carbon dioxide, or carbon monoxide. The deoxygenation reaction for fatty acids and triglycerides are respectively illustrated as:

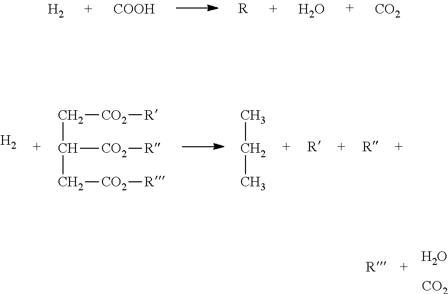

During the deoxygenation reaction, the length of a paraffin chain Rⁿ created will vary by a value of one depending on the exact reaction pathway. It is understood that deoxygenation includes at least one of hydrodeoxygenation, decarboxylation, and decarbonylation, or any combination thereof. For instance, if carbon dioxide is formed, then the chain will have one fewer carbon than the fatty acid source. If water is formed, then the chain will match the length of the fatty acid source.

Operating conditions for the deoxygenating unit include pressures in the range of from about 250 to about 800 psig (about 1724 to about 5516 kPa) and temperatures in the range of from about 274° C. to about 371° C. (about 525° F. to about 700° F.) in one embodiment, from about 274° C. to about 338° C. (about 525° F. to about 640° F.) in another embodiment and from about 274° C. to about 310° C. (about 525° F. to about 590° F.) in another embodiment. Catalysts may include those containing one or more of Ni, Mo, Co, P, such as Ni—Mo, Ni—Mo—P, Ni—Co—Mo, or Co—Mo, on alumina, silica, titania, zirconia, and mixtures thereof. Suitable hydrogen to hydrocarbon mole ratios include from about 1500 to 10,000, from about 4000 to 9000, and from about 5000-8000 standard cubic feet per barrel of feedstock (scf/B). Suitable space velocities include 0.2-3.0 hr⁻¹ LHSV. Conditions are selected to minimize cracking or isomerizing the paraffins.

The deoxygenated product containing normal paraffins, water, carbon dioxide, carbon monoxide, and propane is fractionated into a C9 to C14 stream 115 and a C14+ stream 120. The separation may be performed in a multi-stage fractionation unit, distillation system or similar known apparatus. In any event, the separator removes the water, carbon dioxide, carbon monoxide, and propane from the deoxygenated product. A naphtha stream of paraffins with carbon chain lengths of C₅ to C9 (not shown) may also be formed.

The C14+ stream 120 is sent to the linear selective cracking unit 125 where it is selectively cracked to form a first stream 130 comprising normal or lightly branched C9 to C14 paraffins and a second stream 135 comprising isoparaffins, as described above.

The C9 to C14 stream 115 from the deoxygenation unit 110 and the first stream 130 from the linear selective cracking unit 125 are sent to a decontamination unit 140. The decontamination unit 140 removes contaminants in an adsorption system from the C9 to C14 paraffins in the C9 to C14 stream 115 and the first stream 130. The contaminants include, but are not limited to, sulfur compounds, or nitrogen compounds, or phosphorous compounds, or oxygenates, or aromatics or combinations thereof.

The decontaminated stream 145 is sent to a dehydrogenation unit 150 where hydrogen is removed to produce a dehydrogenated stream 155 comprising mono-olefins, di-olefins, and aromatics. In the dehydrogenation unit 150, the paraffins are dehydrogenated into mono-olefins of the same carbon numbers as the paraffins. Typically, dehydrogenation occurs through known catalytic processes, such as the commercially popular Pacol process. Di-olefins (i.e., dienes) and aromatics are also produced as an undesired result of the dehydrogenation reactions as expressed in the following equations:

Mono-olefin formation:

CₓH₂ₓ₊₂→CₓH₂ₓ+H₂

Di-olefin formation:

CₓH₂ₓ→CₓH₂ₓ₋₂+H₂

Aromatic formation:

CₓH₂ₓ₋₂→CₓH₂ₓ₋₆+2H₂

Operating conditions for the dehydrogenation unit 150 include space velocities from about 5 to about 50 LHSV and from about 20 to about 32 LHSV; pressures from about 1 kPa (g) to about 1013 kPa (g) (about 0.1 psig to about 150 psig); temperatures from about 400° C. to about 500° C. and from about 440° C. to about 490° C., and hydrogen to hydrocarbon mole ratios from about 1-12 and from about 3-7. An example of a suitable catalyst is a Pt on alumina catalyst where platinum is attenuated with an attenuator metal. Another suitable catalyst is described in U.S. Pat. No. 6,177,381 hereby incorporated by reference in its entirety. The dehydrogenation unit 150 may be operated dry or with water injection up to about 2000 mass-ppm water. Hydrogen can be recycled to the deoxygenation unit upstream.

The dehydrogenated stream 155 is sent to a selective hydrogenation unit 160, such as a DeFine reactor, where at least a portion of the di-olefins are hydrogenated to form additional mono-olefins. As a result, the mono-olefin stream 170 has an increased mono-olefin concentration compared to the dehydrogenated stream 155. The aromatics are separated and removed as aromatics stream 165. A light end stream 167 containing any lights, such as butane, propane, ethane and methane, that resulted from cracking or other reactions during upstream processing can also removed.

The mono-olefin stream 170 comprising mono-olefins is sent to the alkylation unit 175 along with a benzene stream 180. The benzene is alkylated with the mono-olefins to form alkylbenzene. The alkylation unit 175 contains a catalyst, such as a solid acid catalyst, that supports alkylation of the benzene with the mono-olefins. Fluorinated silica-alumina, hydrogen fluoride (HF), aluminum chloride (AiCl₃), zeolitic, and ionic liquid catalysts are examples of major catalysts in commercial use for the alkylation of benzene with linear mono-olefins and may be used in the alkylation unit 175. As a result of alkylation, alkylbenzene, typically called linear alkylbenzene (LAB), is formed according to the reaction:

C₆H₆+CₓH₂ₓ→C₆H₅CₓH₂ₓ₊₁

Suitable operating conditions for the alkylation unit 175 include space velocities from 1 to about 10 LHSV, pressures to maintain liquid phase operation, such as about 2068 kPa (g) to about 4137 kPa (g) (about 300 psig to about 600 psig), temperatures in the range of from about 80° C. to about 180° C. and 120° C. to about 170° C., benzene to olefin mole ratios of about 3 to about 40 and about 8 to about 35.

Surplus amounts of benzene are supplied to the alkylation unit 175 to achieve high degree of desired alkylation. Therefore, the alkylation effluent 185 exiting the alkylation unit 175 contains alkylbenzene and unreacted benzene. Further the alkylation effluent 185 may also include some unreacted paraffins. The alkylation effluent 185 is passed to a benzene separation unit 190, such as a fractionation column, for separating the unreacted benzene and paraffins from the alkylation effluent 185. The unreacted benzene exits the benzene separation unit 190 in a benzene recycle stream 195 that may be sent back into the alkylation unit 175 to maintain the desired benzene/olefin ratio (e.g., 1-50) to reduce the volume of fresh benzene needed. The fresh benzene requirement (i.e., the net benzene) is determined by the net olefin to the alkylation unit. A paraffin stream 200 can also be separated out and recycled to the dehydrogenation unit 150.

As a result of the post-alkylation separation processes, the linear alkylbenzene product 205 is isolated. It is noted that such separation processes are not necessary in all embodiments in order to isolate the linear alkylbenzene product 205.

The linear alkylbenzene product 205 is a linear alkylbenzene product comprising: alkylbenzenes having the formula C₆H₅CₓH₂ₙ₊₁ wherein n is from 9 to 14. In some embodiments, at least 80 mass % of the alkylbenzenes have linear alkyl groups, or at least 90 mass %.

The linear alkylbenzene may be sulfonated to provide a linear alkylbenzene sulfonate product comprising: alkylbenzene sulfonate compounds having the formula CₙH₂ₙ₊₁C₆H₄SO₃H wherein n is from 10 to 14, or wherein n is from 11 to 13.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator which may be operated at higher pressure. The term “communication” means that fluid flow is operatively permitted between enumerated components, which may be characterized as “fluid communication”. The term “downstream communication” means that at least a portion of fluid flowing to the subject in downstream communication may operatively flow from the object with which it fluidly communicates.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Unless indicated otherwise, overhead lines and bottoms lines refer to the net lines from the column downstream of any reflux or reboil take-off to the column. Stripper columns may omit a reboiler at a bottom of the column and instead provide heating requirements and separation impetus from a fluidized inert media such as steam.

As used herein, the term “a component-rich stream” or “a component stream” means that the stream coming out of a vessel has a greater concentration of the component than the feed to the vessel. As used herein, the term “a component-lean stream” means that the lean stream coming out of a vessel has a smaller concentration of the component than the feed to the vessel.

### EXAMPLES

### Example 1

A coconut oil feed was deoxygenated, to form paraffins, dehydrogenated to form mono-olefins, and benzene was alkylated with the mono-olefins to form an alkylbenzene product with a modern carbon content of 62 mass 96 modern carbon as determined by ASTM D6866 as compared to a theoretical modern carbon content of 66.4 mass %, a bromine number of 1 g Br/per gram sample as determined by UOP standard test method 304, and a linearity of 92 mass %.

### Example 2

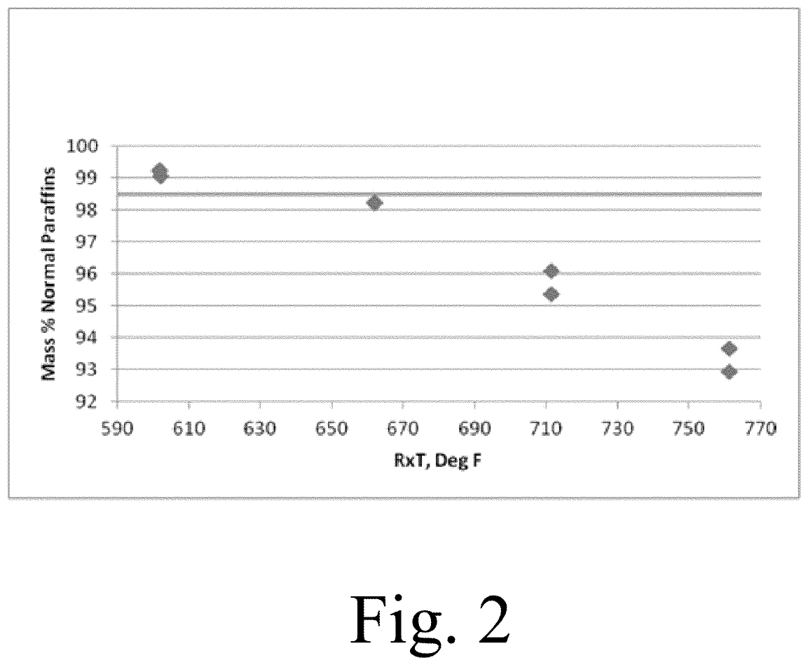

An oil was deoxygenated using a catalyst at a pressure of 480 psig, H, to bio-oil ratio of 7200 scf/B and a LHSV of 1 hr′. During operation, the deoxygenation reaction temperature was increased in steps from 315° C. (600° F.) to 34.9° C. (660° F.) and then to 377° C. (710° F.) and 404° C. (760° F.) to monitor the response of linearity in the final product to reaction temperature. The results are shown in FIG. 2 which is a plot of the concentration in mass % of normal C10-C13 paraffins versus reaction temperature. FIG. 2 clearly demonstrates that as the deoxygenation reaction temperature is increased, the concentration of linear paraffins decreases. Controlling the temperature to less than 404° C. (760° F.) resulted in greater than 92 mass percent linear paraffins.

Note: Examples 1 and 2 were previously included in U.S. Pat. No. 9,079,814 as Examples 3 and 4.

### SPECIFIC EMBODIMENTS

While the following is described in conjunction with specific embodiments, it will be understood that this description is intended to illustrate and not limit the scope of the preceding description and the appended claims.

A first embodiment of the invention is a method for production of a linear alkylbenzene product derived from a triglyceride comprising deoxygenating a triglyceride which produces 60% or more of normal paraffins having less than 16 carbon atoms after deoxygenation to form a paraffin stream comprising 60% or more of normal paraffins having less than 16 carbon atoms; fractionating the paraffin stream to form a C9 to C14 stream comprising C9 to C14 paraffins and a C14+ stream comprising C14+ paraffins; linear selective cracking the C14+ stream in a separate linear selective cracking unit under linear selective cracking conditions in the presence of a linear selective cracking catalyst to form a first stream comprising normal or lightly branched C9 to C14 paraffins and a second stream comprising isoparaffins; removing contaminants from the C9 to C14 stream and the first stream to form a decontaminated stream wherein the contaminates comprise sulfur compounds, or nitrogen compounds, or phosphorous compounds, or oxygenates, or aromatics or combinations thereof; dehydrogenating the decontaminated stream to provide a dehydrogenated stream comprising mono-olefins, di-olefins, and aromatics; selectively hydrogenating the di-olefins in the dehydrogenated stream to form additional mono-olefins, and separating and removing the aromatics from the mono-olefins to form an aromatics stream comprising the aromatics and a mono-olefins stream comprising the mono-olefins; alkylating benzene with the mono-olefins under alkylation conditions to provide an alkylation effluent comprising alkylbenzenes and benzene; isolating the alkylbenzenes to provide the alkylbenzene product derived from the triglyceride. An embodiment of the invention is one, any or all of prior embodiments in this paragraph up through the first embodiment in this paragraph wherein the C9 to C14 stream and the first stream are combined before removing the contaminants from the C9 to C14 stream and the first stream to form the decontaminated stream. An embodiment of the invention is one, any or all of prior embodiments in this paragraph up through the first embodiment in this paragraph wherein the linear selective cracking catalyst comprises ruthenium, platinum, and nickel supported catalyst or mixtures thereof. An embodiment of the invention is one, any or all of prior embodiments in this paragraph up through the first embodiment in this paragraph wherein the linear selective cracking conditions comprise a temperature in a range of 290° C. to 455° C., or a pressure in a range of 2.8 MPa to 17.5 MPa, or combinations thereof. An embodiment of the invention is one, any or all of prior embodiments in this paragraph up through the first embodiment in this paragraph wherein an amount of alkylbenzene product is greater than an amount of alkylbenzene produced in a process without the linear selective cracking step. An embodiment of the invention is one, any or all of prior embodiments in this paragraph up through the first embodiment in this paragraph wherein the alkylbenzene product comprises alkylbenzenes having C9 to C14 chains. An embodiment of the invention is one, any or all of prior embodiments in this paragraph up through the first embodiment in this paragraph wherein the alkylbenzene product comprises alkylbenzenes having C10 to C13 chains. An embodiment of the invention is one, any or all of prior embodiments in this paragraph up through the first embodiment in this paragraph wherein the triglyceride produces 0.1 wt. % to 20 wt. % of normal paraffins having 16 carbon atoms after dehydrogenation. An embodiment of the invention is one, any or all of prior embodiments in this paragraph up through the first embodiment in this paragraph wherein the C14+ stream comprises C16 to C18 paraffins. An embodiment of the invention is one, any or all of prior embodiments in this paragraph up through the first embodiment in this paragraph wherein the triglyceride produces no less than 15 wt. % of normal paraffins having either 12 or 14 carbon atoms after deoxygenation. An embodiment of the invention is one, any or all of prior embodiments in this paragraph up through the first embodiment in this paragraph where in the triglyceride produces no less than 10 wt. % of normal paraffins having 12 carbon atoms after deoxygenation.

A second embodiment of the invention is a method for production of an alkylbenzene product derived from a triglyceride comprising deoxygenating a triglyceride which produces 60% or more of normal paraffins having less than 16 carbon atoms after deoxygenation to form a paraffin stream comprising 60% or more of normal paraffins having less than 16 carbon atoms; fractionating the paraffin stream to form a C9 to C14 stream comprising C9 to C14 paraffins and a C14+ stream comprising C14+ paraffins; linear selective cracking the C14+ stream in a separate linear selective cracking unit under linear selective cracking conditions in the presence of a linear selective cracking catalyst to form a first stream comprising normal or lightly branched C9 to C14 paraffins and a second stream comprising isoparaffins wherein the linear selective cracking catalyst comprises ruthenium, platinum, and nickel supported catalyst or mixtures thereof; removing contaminants from the C9 to C14 stream and the first stream to form a decontaminated stream wherein the contaminates comprise sulfur compounds, or nitrogen compounds, or phosphorous compounds, or oxygenates, or aromatics or combinations thereof; dehydrogenating the decontaminated stream to provide a dehydrogenated stream comprising mono-olefins, di-olefins, and aromatics; selectively hydrogenating the di-olefins in the dehydrogenated stream to form additional mono-olefins, and separating and removing the aromatics from the mono-olefins to form an aromatics stream comprising the aromatics and a mono-olefins stream comprising the mono-olefins; alkylating benzene with the mono-olefins under alkylation conditions to provide an alkylation effluent comprising alkylbenzenes and benzene; isolating the alkylbenzenes to provide the alkylbenzene product derived from the triglyceride; wherein the alkylbenzene product comprises alkylbenzenes having C9 to C14 chains. An embodiment of the invention is one, any or all of prior embodiments in this paragraph up through the second embodiment in this paragraph wherein the C9 to C14 stream and the first stream are combined before removing the contaminants from the C9 to C14 stream and the first stream to form the decontaminated stream. An embodiment of the invention is one, any or all of prior embodiments in this paragraph up through the second embodiment in this paragraph wherein the linear selective cracking conditions comprise a temperature in a range of 290° C. to 455° C., or a pressure in a range of 2.8 MPa to 17.5 MPa, or combinations thereof. An embodiment of the invention is one, any or all of prior embodiments in this paragraph up through the second embodiment in this paragraph wherein an amount of alkylbenzene product is greater than an amount of alkylbenzene produced in a process without the linear selective cracking step. An embodiment of the invention is one, any or all of prior embodiments in this paragraph up through the second embodiment in this paragraph wherein the triglyceride produces 0.1 wt. % to 20 wt. % of normal paraffins having 16 carbon atoms after deoxygenation. An embodiment of the invention is one, any or all of prior embodiments in this paragraph up through the second embodiment in this paragraph wherein the triglyceride produces no less than 15 wt. % of normal paraffins having either 12 or 14 carbon atoms after deoxygenation. An embodiment of the invention is one, any or all of prior embodiments in this paragraph up through the second embodiment in this paragraph where in the triglyceride produces no less than 10 wt. % of normal paraffins having 12 carbon atoms after deoxygenation.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A method for production of a linear alkylbenzene product derived from a triglyceride comprising:
deoxygenating a triglyceride which produces 60% or more of normal paraffins having less than 16 carbon atoms after deoxygenation to form a paraffin stream comprising 60% or more of normal paraffins having less than 16 carbon atoms;
fractionating the paraffin stream to form a C9 to C14 stream comprising C9 to C14 paraffins and a C14+ stream comprising C14+ paraffins;
linear selective cracking the C14+ stream in a separate linear selective cracking unit under linear selective cracking conditions in the presence of a linear selective cracking catalyst to form a first stream comprising normal or lightly branched C9 to C14 paraffins and a second stream comprising isoparaffins;
removing contaminants from the C9 to C14 stream and the first stream to form a decontaminated stream wherein the contaminates comprise sulfur compounds, or nitrogen compounds, or phosphorous compounds, or oxygenates, or aromatics or combinations thereof,
dehydrogenating the decontaminated stream to provide a dehydrogenated stream comprising mono-olefins, di-olefins, and aromatics;
selectively hydrogenating the di-olefins in the dehydrogenated stream to form additional mono-olefins, and separating and removing the aromatics from the mono-olefins to form an aromatics stream comprising the aromatics and a mono-olefins stream comprising the mono-olefins;
alkylating benzene with the mono-olefins under alkylation conditions to provide an alkylation effluent comprising alkylbenzenes and benzene; and
isolating the alkylbenzenes to provide the alkylbenzene product derived from the triglyceride.

2. The method of claim 1 wherein the C9 to C14 stream and the first stream are combined before removing the contaminants from the C9 to C14 stream and the first stream to form the decontaminated stream.

3. The method of claim 1 wherein the linear selective cracking catalyst comprises ruthenium, platinum, and nickel supported catalyst or mixtures thereof.

4. The method of claim 1 wherein the linear selective cracking conditions comprise a temperature in a range of 290° C. to 455° C., or a pressure in a range of 2.8 MPa to 17.5 MPa, or combinations thereof.

5. The method of claim 1 wherein an amount of alkylbenzene product is greater than an amount of alkylbenzene produced in a process without the linear selective cracking step.

6. The method of claim 1 wherein the alkylbenzene product comprises alkylbenzenes having C9 to C14 chains.

7. The method of claim 1 wherein the alkylbenzene product comprises alkylbenzenes having C10 to C13 chains.

8. The method of claim 1 wherein the triglyceride produces 0.1 wt. % to 20 wt. % of normal paraffins having 16 carbon atoms after deoxygenation.

9. The method of claim 1 wherein the C14+ stream comprises C16 to C18 paraffins.

10. The method of claim 1 wherein the triglyceride produces no less than 15 wt. % of normal paraffins having either 12 or 14 carbon atoms after deoxygenation.

11. The method of claim 1 where in the triglyceride produces no less than 10 wt. % of normal paraffins having 12 carbon atoms after deoxygenation.

12. A method for production of an alkylbenzene product derived from a triglyceride comprising:
deoxygenating a triglyceride which produces 60% or more of normal paraffins having less than 16 carbon atoms after deoxygenation to form a paraffin stream comprising 60% or more of normal paraffins having less than 16 carbon atoms;
fractionating the paraffin stream to form a C9 to C14 stream comprising C9 to C14 paraffins and a C14+ stream comprising C14+ paraffins;
linear selective cracking the C14+ stream in a separate linear selective cracking unit under linear selective cracking conditions in the presence of a linear selective cracking catalyst to form a first stream comprising normal or lightly branched C9 to C14 paraffins and a second stream comprising isoparaffins wherein the linear selective cracking catalyst comprises ruthenium, platinum, and nickel supported catalyst or mixtures thereof;
removing contaminants from the C9 to C14 stream and the first stream to form a decontaminated stream wherein the contaminates comprise sulfur compounds, or nitrogen compounds, or phosphorous compounds, or oxygenates, or aromatics or combinations thereof,
dehydrogenating the decontaminated stream to provide a dehydrogenated stream comprising mono-olefins, di-olefins, and aromatics;
selectively hydrogenating the di-olefins in the dehydrogenated stream to form additional mono-olefins, and separating and removing the aromatics from the mono-olefins to form an aromatics stream comprising the aromatics and a mono-olefins stream comprising the mono-olefins;
alkylating benzene with the mono-olefins under alkylation conditions to provide an alkylation effluent comprising alkylbenzenes and benzene; and
isolating the alkylbenzenes to provide the alkylbenzene product derived from the triglyceride;
wherein the alkylbenzene product comprises alkylbenzenes having C9 to C14 chains.

13. The method of claim 12 wherein the C9 to C14 stream and the first stream are combined before removing the contaminants from the C9 to C14 stream and the first stream to form the decontaminated stream.

14. The method of claim 12 wherein the linear selective cracking conditions comprise a temperature in a range of 290° C. to 455° C., or a pressure in a range of 2.8 MPa to 17.5 MPa, or combinations thereof.

15. The method of claim 12 wherein an amount of alkylbenzene product is greater than an amount of alkylbenzene produced in a process without the linear selective cracking step.

16. The method of claim 12 wherein the triglyceride produces 0.1 wt. % to 20 wt. % of normal paraffins having 16 carbon atoms after deoxygenation.

17. The method of claim 12 wherein the triglyceride produces no less than 15 wt. % of normal paraffins having either 12 or 14 carbon atoms after deoxygenation.

18. The method of claim 12 where in the triglyceride produces no less than 10 wt. % of normal paraffins having 12 carbon atoms after deoxygenation.
